(19) United States
(12) Patent Application Publication
(10) Pub. No.: US 2005/0101814 A1
(43) Pub. Date: May 12, 2005

(54) Ring opening for increased olefin production

(76) Inventors: Timothy D. Foley, Forest Park, IL (US); Leonid B. Galperin, Wilmette, IL (US); Michael J. McCall, Geneva, IL (US); Joseph A. Kocal, Glenview, IL (US); Bryan K. Glover, Algonquin, IL (US); Peter R. Pujado, Kildeer, IL (US)

Correspondence Address: JOHN G TOLOMEI, PATENT DEPARTMENT;UOP LLC, 25 EAST ALGONQUIN ROAD, P O BOX 5017, DES PLAINES, IL, 60017-5017, US

(21) Appl. No.: 10/703,771

(22) Filed: Nov. 7, 2003

(51) Int. Cl. C07C 2/00

(52) U.S. Cl. 585/317

(57) ABSTRACT

A process for cracking a naphtha feedstream to light olefins is presented. The process comprises converting aromatics and naphthenes to paraffins, and separating iso- and normal paraffins using a ring opening reactor and an adsorption separation unit.

### FIELD OF THE INVENTION

The present invention relates to a process for the production of light olefins from a naphtha feed stream.

### BACKGROUND OF THE INVENTION

Ethylene and propylene, light olefin hydrocarbons with two or three carbon atoms per molecule, respectively, are important chemicals for use in the production of other useful materials, such as polyethylene and polypropylene. Polyethylene and polypropylene are two of the most common plastics found in use today and have a wide variety of uses for, for example, a material for fabrication and as a material for packaging. Other uses for ethylene and propylene include the production of vinyl chloride, ethylene oxide, ethylbenzene and alcohol. Steam cracking, or pyrolysis, of hydrocarbons produces essentially all of the ethylene and propylene. Hydrocarbons used as feedstock for light olefin production include natural gas, petroleum liquids, and carbonaceous materials including coal, recycled plastics or any organic material. An important feedstream is a naphtha feedstream which is produced during the fractionation of crude oil.

### SUMMARY OF THE INVENTION

The present invention is a process for converting a naphtha feedstream to light olefins. The process uses an adsorptive separation unit which reduces the cost of separating normal paraffins from a naphtha hydrocarbon fraction. The process produces a first process stream comprising primarily n-paraffins, and a second process stream comprising non-normal hydrocarbons. The second process stream is processed through a ring opening reactor that hydrogenates and converts the aromatics and naphthenes to paraffins. The paraffins from the adsorptive separation unit and the hydrogenation ring opening reactor are then passed through a steam cracking unit to produce light olefins. This process increases the yield of light olefins from a naphtha feedstream. The process may optionally include the passing of a py-gas stream generated in the steam cracking unit to the ring opening reactor to further increase the light olefin production.

In an alternate process of the present invention, a ring opening reactor is used to hydrogenate and convert aromatics and naphthenes to paraffins producing a paraffin stream. The paraffin stream is separated in a fractionation unit to separate the normal paraffins from the isoparaffins, where the normal paraffins are sent to a steam cracking unit for the production of light olefins. The iso-paraffins are passed to an isomerization unit for the conversion of a portion of the iso-paraffins to normal paraffins, and the resulting mixture is recycled to the adsorption unit. The isomerization unit further increases light olefin production by increasing the amount of normal paraffins recovered from the naphtha feed stream.

Other objects, advantages and applications of the present invention will become apparent to those skilled in the art from the following detailed description.

### BRIEF DESCRIPTION OF THE DRAWINGS

FIG. 1 is a simplified process flow diagram showing an embodiment for producing light olefins from a naphtha feedstream;

FIG. 2 is an alternate process flow diagram showing an embodiment for producing light olefins from a naphtha feedstream;

FIG. 3 is a process flow diagram showing an embodiment for producing light olefins from a naphtha feedstream, while first processing the feedstream through a ring opening reactor;

FIG. 4 is an alternate process flow diagram showing an embodiment for producing light olefins from a naphtha feedstream, while first processing the feedstream through a ring opening reactor;

FIG. 5 is simplified process for producing light olefins from a naphtha feedstream by processing the feedstream through a ring opening reactor;

FIG. 6 is an embodiment of the invention without the depentanizer; and

FIG. 7 shows the product composition from the ring opening reactor.

### DETAILED DESCRIPTION OF THE INVENTION

The great bulk of the ethylene consumed in the production of various plastics and petrochemicals such as polyethylene is produced by the thermal cracking of higher molecular weight hydrocarbons. Steam is usually mixed with the feed stream to the cracking reactor to reduce the hydrocarbon partial pressure and enhance olefin yield and to reduce the formation and deposition of carbonaceous material in the cracking reactors. The process is therefore often referred to a steam cracking or pyrolysis.

The composition of the feed to the steam cracking reactor affects the results. A fundamental basis of this is the propensity of some hydrocarbons to crack more easily than others. The normal ranking of hydrocarbons' tendency to crack to light olefins is normally given as normal paraffins; isoparaffins; olefins; naphthenes; and aromatics. Benzene and other aromatics are particularly resistant to steam cracking and undesirable as cracking feedstocks, with only the alkyl sidechains being cracked to produce the desired product. The feed to a steam cracking unit is normally a mixture of hydrocarbons varying both by type of hydrocarbon and carbon number. This variety results in it being very difficult to separate less desirable feed components, such as naphthenes and aromatics, from the feedstream by fractional distillation. The hydrocarbons that are not the normal paraffins can be removed by solvent extraction or adsorption. These hydrocarbons can be upgraded to improve the feedstock to the steam cracking unit. The present invention provides a process for converting the aromatics and naphthenes to paraffins, and separating paraffins to be passed to a steam cracking unit.

The feedstream to a steam cracking unit can be quite diverse and can be chosen from a variety of petroleum fractions. The feedstream to the subject process preferably has a boiling point range falling within the naphtha boiling point range or about 36 to 205° C. It is preferred that the feed stream does not contain appreciable amounts, e.g. more than 5 mole %, of C₁₂ hydrocarbons. A representative feed stream to the subject process is a C₅-C₁₁ fraction produced by fractional distillation of a hydrotreated petroleum fraction. Hydrotreating is desired to reduce the sulfur and nitrogen content of the feed down to acceptable levels. A second representative feed is a similar fraction comprising C₅ through C₉ hydrocarbons. The feed will preferably have a carbon number range of at least three. It is within the scope of the subject invention that the feed stream to the process comprise primarily the heavier C₆-plus hydrocarbons. In this case the lightest (most volatile) hydrocarbons, the C₅ hydrocarbons, are concentrated into a stream which is used as the desorbent in an adsorptive separation zone. The light fraction employed as the desorbent preferably contains essentially only hydrocarbons having the same carbon number e.g. C₅ or C₆ hydrocarbons. This light fraction will contain a variety of hydrocarbon types, but preferably contains at least 90 mol-% of the same carbon number.

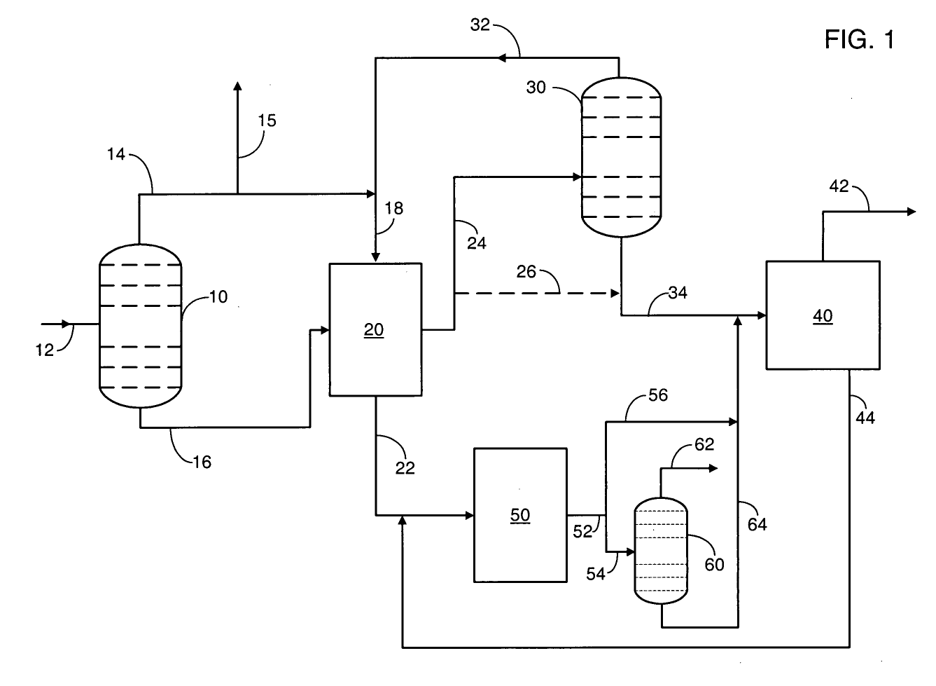

In one embodiment, the process is shown in FIG. 1. A full naphtha boiling range feedstream enters the process through line 12. The feedstream is passed into a first fractionation unit 10. This fractionation unit 10 is a distillation unit and is designed and operated to function as a depentanizer separating the entering hydrocarbons into a first process stream leaving through line 14, which is rich in C₅ paraffins, and a second process stream leaving through line 16, which comprises C₆ and greater hydrocarbons. It is preferable that the C₅ hydrocarbons be substantially removed from the hydrocarbons in the second process stream, as the C₅ hydrocarbons are to be used as the desorbent in a unit downstream of the first fractionation unit 10.

The second process stream is passed into an adsorption separation unit 20. The adsorption separation unit 20 may be of any suitable type that is appropriate for the specific situation of the process. The adsorption unit 20 is comprised of a bed of adsorbent comprised of a molecular sieve or other appropriate adsorbent for adsorbing hydrocarbons. Examples of suitable adsorption separation units include, but are not limited to, swing bed or simulated moving bed adsorption units. The second process stream is separated in the adsorption unit 20 by the selective adsorption and retention of normal paraffins in the adsorption bed. The adsorption separation process undergoes an adsorption step, wherein selected components of the second process stream are adsorbed onto the adsorbent, and followed by a desorption step wherein the selected components are desorbed from the adsorbent. In this case, the selected components are the normal paraffins in the second process stream. The normal paraffins remain on the adsorbent until a desorbent is passed through the adsorption unit 20.

During the adsorption step, the normal paraffins are separated from the second process stream by adsorption onto the adsorbent. The remaining components of the second process stream are non-normal hydrocarbons and pass through the adsorption bed unaffected. The non-normal hydrocarbons pass out of the adsorption unit 20 as a raffinate stream via line 22.

During the desorption step, a desorbent is delivered to the adsorption unit 20 through line 18 and passes through the adsorbent bed. The desorbent has properties which enable it to displace the heavier normal paraffins from the adsorbent, resulting in the formation of an extract stream. The extract stream comprises C₆ through C₁₁ normal hydrocarbons and a portion of the desorbent material, or in this case, C₅ hydrocarbons. The extract stream leaves the adsorption unit 20 through line 24 and passes to a second fractionation unit 30. The second fractionation unit 30 is also referred to as the extract column. The second fractionation unit 30 is designed and operated to separate the desorbent from the C₆ through C₁₁ normal paraffins, producing an overhead desorbent stream and a bottoms extract stream of C₆ through C₁₁ normal paraffins. The desorbent stream is recycled from the second fractionation unit 30 through line 32 to the adsorption unit 20. The extract stream is passed through line 34 to a steam cracking unit 40. The steam cracking unit 40 is operated at steam cracking conditions effective to convert the paraffins into a stream comprising predominantly ethylene and propylene. The ethylene and propylene stream is removed from the steam cracking unit 40 through line 42.

While the overhead stream of line 14 is passed to the adsorption unit 20 as desorbent, the continuous process of recovery and recycling of desorbent means that some of the desorbent must be rejected from the process. One optional way of diverting some of the desorbent is for the process to have a C₅ bleed line from the process through line 15.

The raffinate stream, produced during the adsorption step, is passed through line 22 to a ring opening reactor 50. The ring opening reactor 50 processes the raffinate stream by opening the naphthene rings and converts the naphthenes to paraffins. Preferably, the ring opening reactor 50 includes hydrogenation for converting aromatic compounds to naphthenes. Hydrogen is supplied to the ring opening reactor 50 for the hydrogenation. One source of hydrogen available to use in the ring opening reactor 50 is from the steam cracking unit 40. The steam cracking unit 40 generates hydrogen as a byproduct of the cracking process. The converted naphthenes are subsequently opened by the ring opening catalyst. The ring opening reactor 50 comprises a catalyst in a catalyst bed over which the raffinate stream flows. The ring opening reactor 50 produces a ring opening process stream that passes through line 52. The ring opening process stream can be passed to the steam cracking unit 40 for conversion of the paraffins to ethylene and propylene.

The ring opening process stream can include methane (CH₄) depending on the composition of the raffinate stream, and the reaction conditions of the ring opening reactor 50. The methane takes up energy and space within the stream cracking unit, and can interfere with the steam cracking process of the paraffins, without contributing to the production of ethylene. When the conditions produce methane, it is desirable to remove the methane before passing the ring opening process stream to the steam cracking unit 40. The ring opening process stream is passed through line 54 to an optional demethanizer, or third fractionation unit 60. The third fractionation unit 60 separates the ring opening process stream into a methane stream and a paraffin process stream comprising normal and iso-paraffins. The methane stream is removed overhead through line 62, and the paraffin process stream is passed through line 64 to the steam cracking unit 40.

The steam cracking unit 40, in addition to generating light olefins, generates a by-product known as pyrolysis gasoline (py-gas). The py-gas is a mixture of light hydrocarbons and includes benzene, toluene, other aromatics, and naphthenes. The py-gas is separated from the ethylene by a water quench stage. The py-gas is passed through line 44 leaving the steam cracking unit 40 to the ring opening reactor 50. The py-gas recycling provides an additional increase in the light olefin production from a naphtha feedstream in the present process.

The raffinate stream may also contain some C₅ hydrocarbons which previously occupied the void spaces of the adsorbent bed(s) through which it has passed. Optionally, the raffinate stream can be passed to a fourth fractionation unit (not shown) before passing the raffinate stream to the ring opening reactor 50. The fourth fractionation unit is a depentanizer and is for separating the C₅ hydrocarbons that are passed out of the adsorption unit 20 with the raffinate stream. The fourth fractionation unit produces a C₅ rich stream which is recycled to be reused as the desorbent, and a non-normal hydrocarbon stream which is passed to the ring opening reactor 50.

This system is usually set up with a series of adsorbent units 20 such that the system can be run on a continuous basis wherein the first and second process streams are directed to different adsorbent units 20 at different times.

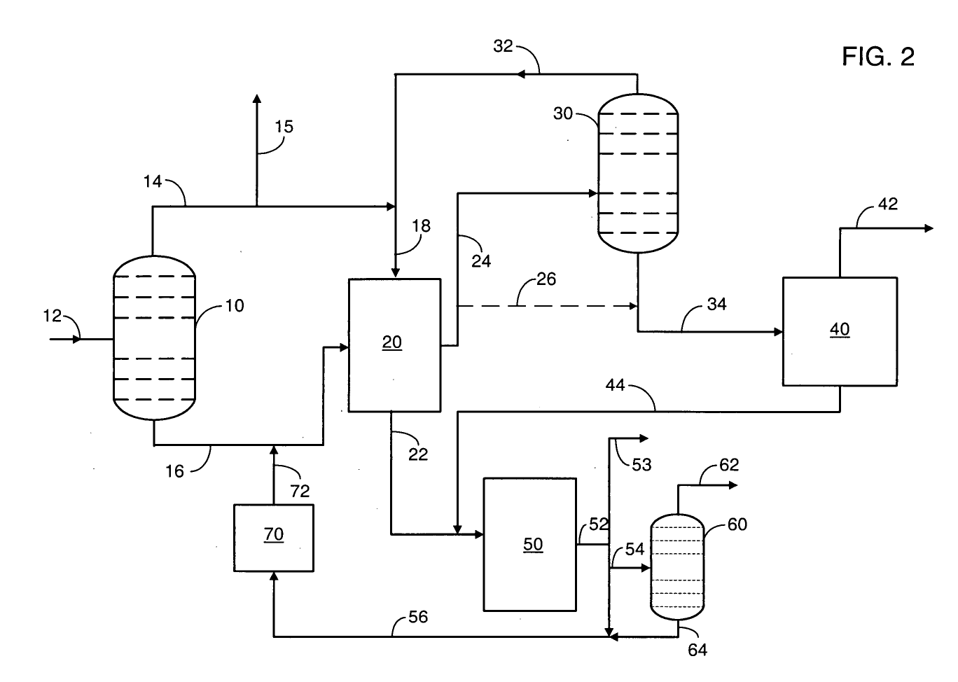

An alternate embodiment of the present invention is shown in FIG. 2. As with the first embodiment, a naphtha feedstream enters a first fractionation unit 10 through line 12. The feedstream is split into a first process stream comprising primarily C₅ hydrocarbons leaving through line 14, and a second process stream comprising C₆ through C₁₁ hydrocarbons leaving through line 16. The second process stream is passed to an adsorption separation unit 20. The adsorption unit 20 adsorbs the C₆ through C₁₁ normal paraffins and produces a raffinate stream that leaves through line 22. As with the first embodiment, the first process stream enters the adsorption unit 20 through line 18 and produces an extract stream comprising C₆ through C₁₁ normal paraffins and C₅ hydrocarbons. The extract stream passes through line 24 to a second fractionation unit 30 producing a third process stream comprising the C₅ hydrocarbons and a fourth process stream comprising the C₆ through C₁₁ normal paraffins. The third process stream is passed through line 32 to recycle the C5 hydrocarbons as desorbent, and the fourth process stream is passed through line 34 to a steam cracking unit 40.

The raffinate stream is passed to a ring opening reactor 50, wherein the raffinate stream undergoes hydrogenation of the aromatics to naphthenes and paraffins, and the naphthenes are converted to normal and iso-paraffins, producing a ring opening process stream. At least a portion of the ring opening process stream is passed through line 52 to line 56 where the ring opening process stream is recycled to the adsorption unit 20. The ring opening reactor 50 generates a mixture of normal and iso-paraffins, and recycling the ring opening process stream to the adsorption unit 20 increases the recovery of normal paraffins to pass to the steam cracking unit 40. As the process of recycling the ring opening process stream would create a buildup of iso-paraffins, some of the ring opening process stream is diverted to other places through line 53.

The steam cracking unit 40 creates py-gas which is separated from the light olefins. The py-gas is passed through line 44 to the ring opening reactor 50, where the py-gas is hydrogenated and the naphthenes are opened to form normal and iso-paraffins. The treated py-gas is processed to be recycled to the steam cracking unit 40 to further increase the light olefin yield from the naphtha feedstream.

The ring opening process stream may comprise some methane and light hydrocarbons (C2 through C4). The methane may be generated as a result of a combination of raffinate stream compositions and operating conditions of the ring opening reactor 50. If methane is generated, the methane may optionally be removed. Removing these light hydrocarbons, and especially the methane will improve the efficiency of the adsorption unit 20 and the steam cracking unit 40. The ring opening process stream optionally passes through line 52 to line 54 and passes into a demethanizer fractionation unit 60. The demethanizer 60 separates the ring opening process stream into a methane rich process stream, and a demethanized process stream comprising normal and isoparaffins. The methane rich process stream is passed through line 62. The demethanized process stream passes to the adsorption unit 20 through line 64.

An optional process in this embodiment includes the use of an isomerization unit 70. The isomerization unit 70 will generate an equilibrium mixture of normal and iso-paraffins. The ring opening process stream will pass to the isomerization unit 70, and the isomerization unit will generate an isomerized process stream, comprising roughly equivalent amounts of normal and iso-paraffins by weight. Depending on the amount of methane produced in the ring opening reactor 50, the ring opening process stream can go directly to the isomerization unit 70, or first through the demethanizer fractionation unit 60. The isomerized process stream will pass through line 72 to the adsorption unit 20, where the normal paraffins are adsorbed and produce a raffinate stream rich in iso-paraffins. The iso-paraffins after passing through the ring opening reactor 50 are recycled to the isomerization unit 70 and will have a portion of the iso-paraffins converted to normal paraffins.

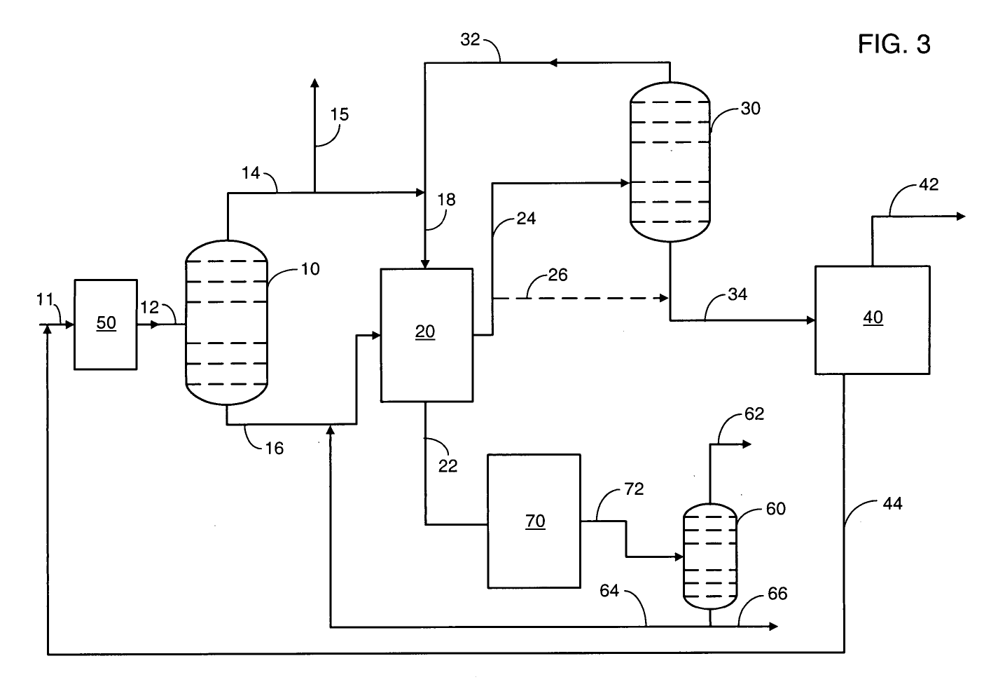

A third embodiment entails passing the naphtha feedstream to a ring opening reactor 50 through line 11, and is shown in FIG. 3. The ring opening reactor 50 produces a ring opening process stream that passes to a first fractionation unit 10 through line 12. The ring opening reactor 50 preferably further includes a hydrogenation function for hydrogenating aromatics in the naphtha feedstream. The ring opening reactor 50 also cleaves the naphthene rings and increases the amount of normal and iso-paraffins in the ring opening process stream. The first fractionation unit 10 separates the ring opening process stream into a first process stream rich in C₅ hydrocarbons and a second process stream comprising C₆ through C₁₁ hydrocarbons. The first process stream leaves the first fractionation unit 10 through line 14. The second process stream passes through line 16 to an adsorption unit 20, where the normal paraffins are adsorbed, and forms a raffinate stream comprising iso-paraffins and other non-normal paraffins. The raffinate stream is drawn off through line 22.

A portion of the first process stream enters the adsorption unit through line 18 and acts as a desorbent displacing the adsorbed normal paraffins in the adsorption unit 20, forming an extract stream comprising the normal paraffins and desorbent. The extract stream is passed through line 24 to a second fractionation unit 30, where the normal paraffins are separated from the desorbent forming a desorbent stream and a normal paraffin stream. The desorbent stream is drawn off through line 32 and recycled to the adsorption unit 20 through line 18. The normal paraffins are passed from the second fractionation unit 30 through line 34 to a steam cracking unit 40, where the paraffins are cracked to form light olefins. Optionally, depending on the desorbent content in the extract stream, the extract stream can be directed to the steam cracking unit 40 directly through line 26.

The steam cracking unit 40, in addition to generating light olefins, generates a py-gas. The py-gas is directed to the ring opening reactor 50 through line 44. The py-gas is substantially converted to normal and iso-paraffins, thereby increasing the light olefin yield from the naphtha feed stream.

The raffinate stream is passed through line 22 to an isomerization unit 70. The isomerization unit isomerizes the iso-paraffin rich raffinate stream to a mixture of iso-paraffins and normal paraffins and forms an isomerization stream. The isomerization stream passes through line 72 to a third fractionation unit 60. The third fractionation unit 60 separates C₅ and lighter hydrocarbons for recycle to the adsorption unit 20, or redirection for other processing that is passed through line 62, and an isomerization recycle stream that is passed through line 64. The isomerization recycle stream comprises a mixture of normal and iso-paraffins, and is passed to the adsorption unit 20. The conversion of iso-paraffins from the adsorption unit 20 to a mixture of iso-paraffins and normal paraffins in the isomerization unit 70 improves the overall conversion of the naphtha feedstream to light olefins.

Optionally, a portion of the isomerization recycle stream can be bled off through line 66 and routed to other units, including passing some of the isomerization recycle stream to the steam cracking unit 40.

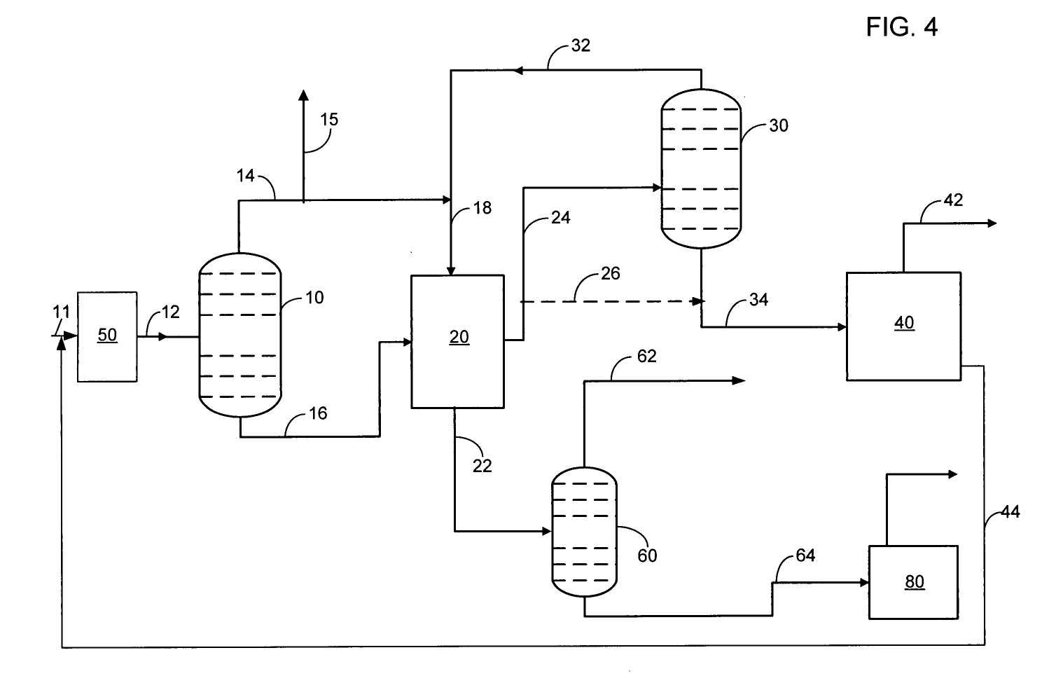

A fourth embodiment of the present invention is shown in FIG. 4. A naphtha feedstream is passed through line 11 to a ring opening reactor 50. The naphtha feedstream is hydrogenated and the naphthenes rings are opened to convert aromatics and naphthenes to paraffins, generating a ring opening product stream that is rich in paraffins. The ring opening product stream is passed through line 12 to a first fractionation unit 10. The first fractionation unit 10 separates the feedstream into a first process stream that comprises primarily C₅ and lighter hydrocarbons, and a second process stream that comprises C₆ through C₁₁ hydrocarbons. The second process stream passes through line 16 to an adsorption unit 20. The adsorption unit 20 separates and retains the normal paraffins from the second process stream producing a raffinate stream comprising non-normal hydrocarbons. The raffinate stream passes through line 22 to a third fractionation unit 60. The third fractionation unit 60 separates C₅ hydrocarbons from the raffinate stream which pass through line 62. The C₅ hydrocarbon stream can be recycled to the adsorption unit 20. The third fractionation unit 60 also generates a bottoms stream comprising C₆ through C₁₁ non-normal hydrocarbons. The bottoms stream passes through line 64 to further processing units, such as for example a reformer 80.

The first process stream passes through line 14 to line 18 and enters the adsorption unit 20. The first process stream acts as a desorbent and displaces the normal paraffins that have been removed from the second process stream, generating an extract stream comprising C₆ through C₁₁ normal paraffins and a portion of the C₅ hydrocarbons from the desorbent. The extract stream passes through line 24 to a second fractionation unit 30, wherein the C₅ hydrocarbons are removed through line 32 and recycled to be reused as desorbent. The second fractionation unit 30 also produces a paraffin stream, that passes through line 34 to a steam cracking unit 40. The steam cracking unit 40 converts the normal paraffins to a light olefin product stream that passes out through line 42. The steam cracking unit 40 also generates a py-gas stream. The py-gas is recycled through line 44 to the ring opening reactor 50 for hydrogenation and ring opening.

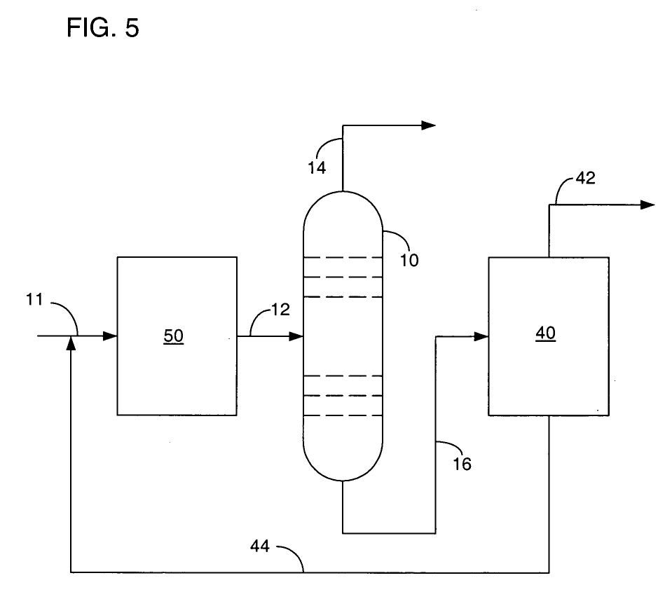

A fifth embodiment of the present invention is shown in FIG. 5. This embodiment makes use of the ability to crack iso-paraffins in a steam cracking unit 40 to generate light olefins, and uses a minimum of equipment in conjunction with a ring opening reactor 50. As naphtha feedstreams from different oil sources generate different overall compositions, and for a feedstream that can be substantially converted to normal and iso-paraffins through hydrogenation and ring opening, the naphtha feedstream can be processed without an adsorption unit. The ring opening product stream is passed through line 12 to a steam cracking unit 40, where the paraffins are converted to light olefins. Optionally, the ring opening product stream is passed through line 12 to a first fractionation unit 10. The first fractionation unit 10 separates the ring opening product stream into a first process stream comprising methane, and a second process stream comprising substantially C₂ and higher normal and iso-paraffins. The second process stream passes through line 16 to the steam cracking unit 40, where the paraffins are converted to light olefins. The steam cracking unit 40 also generates a py-gas, which recycles through line 44 to the ring opening reactor 50.

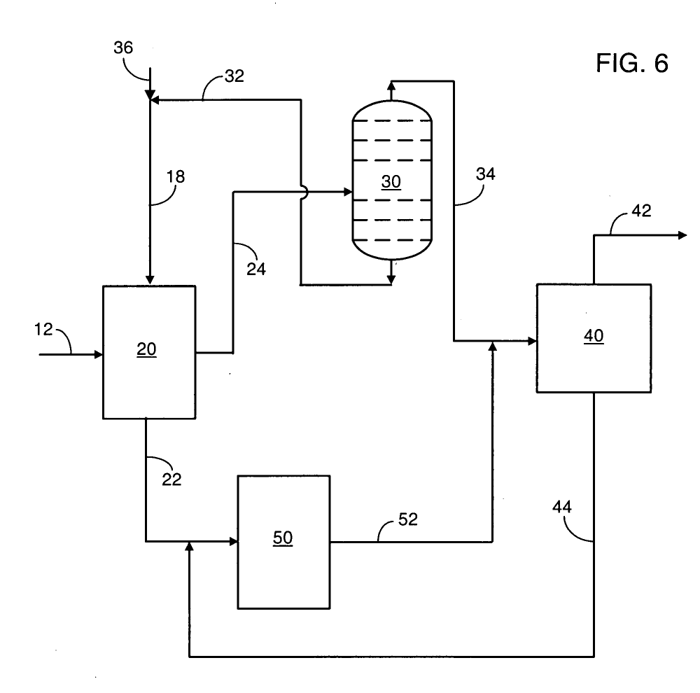

The invention allows for many variations, and as such allows for alternate desorbent materials. In one embodiment, as shown in FIG. 6, feed stream is passed through line 12 to an adsorption unit 20. The adsorption unit 20 separates and retains the normal paraffins from the feedstream, producing a raffinate stream comprising non-normal hydrocarbons.

A desorbent passes through line 18 and enters the adsorption unit 20. The desorbent is a heavy normal hydrocarbon relative to the normal hydrocarbons in the naphtha feedstream. For a naphtha feedstream with hydrocarbons in the C₅ through C₁₁ range, a preferred heavy normal hydrocarbon is normal dodecane, or a normal C₁₂ hydrocarbon. The choice of heavy normal hydrocarbon is dependant as the feedstream, and for a feedstream with larger hydrocarbon molecules, a heavy normal hydrocarbon having greater than 12 carbons is desired. The desorbent displaces the normal hydrocarbons and produces an extract stream. The extract stream passes through line 24 to a fractionation unit 30. The fractionation unit 30 separates the extract stream into a normal hydrocarbon stream and a desorbent stream. The desorbent stream is passed through line 32 and recycled for use in the adsorbent unit 20. The normal hydrocarbon stream is passed through line 34 to a steam cracking unit 40, wherein the normal paraffins are converted to ethylene and propylene. Optionally, when desorbent is consumed in the process, additional desorbent can be added through line 36.

The raffinate stream passes through line 22 to a ring opening reactor 50. The ring opening reactor 50 processes the raffinate stream by hydrogenating aromatic compounds to naphthene ring compounds, and opening naphthene ring compounds to paraffins. The ring opening reactor 50 produces a ring opening process stream comprising paraffins that passes through line 52. The ring opening process stream passes to the steam cracking unit 40 where the paraffins are converted to ethylene and propylene, which are passed through line 42.

The steam cracking unit 40 produces py-gas as a byproduct which has an aromatic content. Optionally, the py-gas is passed through line 44 to the ring opening unit 50, where the py-gas undergoes hydrogenation and ring opening to convert the py-gas to a stream rich in paraffins.

Optionally, in this embodiment, the ring opening process stream may be passed to an isomerization unit (not shown) where a stream rich in iso-paraffins is converted to a stream with a mixture of normal and iso-paraffins. The normal and iso-paraffins are then passed to the steam cracking unit 40.

Another option with this embodiment includes passing a portion of the ring opening process stream to the adsorption unit 20. This further separates the normal and iso-paraffins for sending a greater amount of normal paraffins to the steam cracking unit 40 while recycling the non-normal hydrocarbons.

The naphtha feedstock generally contains small amounts of sulfur compounds amounting to less than 10 mass parts per million (ppm) on an elemental basis. Preferably the naphtha feedstock has been prepared from a contaminated feedstock by a conventional pretreating step such as hydrotreating, hydrorefining or hydrodesulfrization to convert such contaminants as sulfurous, nitrogenous and oxygenated compounds to H₂S, NH₃ and H₂O, respectively, which can be separated from hydrocarbons by fractionation. This conversion preferably will employ a catalyst known to the art comprising an inorganic oxide support and metals selected from Groups VIB(6) and VIII(9-10) of the Periodic Table. Preferably, the pretreating step will provide the process combination with a hydrocarbon feedstock having low sulfur levels disclosed in the prior art as desirable, e.g., 1 mass ppm to 0.1 ppm (100 ppb).

Each of the fractionation zones employed in the process preferably comprises a single fractional distillation column. The fractionation or splitting of the various process streams can, however, be performed in other suitable equipment if desired. As noted earlier, the complete recovery of C₅ hydrocarbons, or other light hydrocarbon, overhead from all three fractionation zones will result in a surplus of C₅ hydrocarbons and a need to draw some of them out of the process. The most common drawoffs will be from the first and/or second fractionation units 10, 30. An alternative is to allow some of the C₅ hydrocarbons to exit the process in the extract and or raffinate streams. This can be done by adjustment of the operation of the fractionation zones or by the use of an inherently less exact separation. The use of a simple flash zone or of a refluxed flash zone is one example of this optional alternative C₅ rejection technique. This not only directs this light material to a suitable hydrocarbon consuming process, but also reduces the overall capital and operating costs of the feed preparation.

The adsorption-separation step of the subject process can be performed in a single large bed of adsorbent or in several parallel beds on a swing bed basis. However, it has been found that simulated moving bed adsorptive separation provides several advantages such as high purity and recovery. Therefore, many commercial scale petrochemical separations especially for the recovery of mixed paraffins are performed using simulated countercurrent moving bed (SMB) technology. The previously cited references are incorporated for their teaching on the performance of this process. Further details on equipment and techniques for operating an SMB process may be found in U.S. Pat. Nos. 3,208,833; 3,214,247; 3,392,113; 3,455,815; 3,523,762; 3,617,504; 4,006,197; 4,133,842; and 4,434,051, all of which are incorporated by reference in their entirety. A different type of simulated moving bed operation which can be performed using similar equipment, adsorbent and conditions but which simulates cocurrent flow of the adsorbent and liquid in the adsorption chambers is described in U.S. Pat. Nos. 4,402,832 and 4,498,991, which are incorporated by reference in their entirety.

Operating conditions for the adsorption chamber used in the subject invention include, in general, a temperature range of from about 20 to about 250° C., with from about 60 to about 200° C. being preferred. Temperatures from 90° C. to 160° C. are highly preferred. Adsorption conditions also preferably include a pressure sufficient to maintain the process fluids in liquid phase; which may be from about atmospheric to about 600 psig. Desorption conditions generally include the same temperatures and pressure as used for adsorption conditions. It is generally preferred that an SMB process is operated with an A:F flow rate through the adsorption zone in the broad range of about 1:1 to 5:0.5 where A is the volume rate of “circulation” of selective pore volume and F is the feed flow rate. The practice of the subject invention requires no significant variation in operating conditions or desorbent composition within the adsorbent chambers. That is, the adsorbent preferably remains at the same temperature throughout the process during both adsorption and desorption.

The adsorbent used in the first adsorption zone preferably comprises aluminosilicate molecular sieves having relatively uniform pore diameters of about 5 angstroms. This is provided by commercially available type 5A molecular sieves produced by UOP LLC.

A second adsorbent which could be used in the adsorption zone comprises silicalite. Silicalite is well described in the literature. It is disclosed and claimed in U.S. Pat. No. 4,061,724 issued to Grose et al., which is incorporated by reference in its entirety. A more detailed description is found in the article, “Silicalite, A New Hydrophobic Crystalline Silica Molecular Sieve,” Nature, Vol. 271, Feb. 9, 1978 which is incorporated herein by reference for its description and characterization of silicalite. Silicalite is a hydrophobic crystalline silica molecular sieve having intersecting bent-orthogonal channels formed with two cross-sectional geometries, 6 Å circular and 5.1-5.7 Å elliptical on the major axis. This gives silicalite great selectivity as a size selective molecular sieve. Due to its aluminum free structure composed of silicon dioxide, silicalite does not show ion-exchange behavior. Silicalite is also described in U.S. Pat. Nos. 5,262,144; 5,276,246 and 5,292,900, which are incorporated by reference in their entirety. These basically relate to treatments which reduce the catalytic activity of silicalite to allow its use as an adsorbent.

The active component of the adsorbent is normally used in the form of particle agglomerates having high physical strength and attrition resistance. The agglomerates contain the active adsorptive material dispersed in an amorphous, inorganic matrix or binder, having channels and cavities therein which enable fluid to access the adsorptive material. Methods for forming the crystalline powders into such agglomerates include the addition of an inorganic binder, generally a clay comprising a silicon dioxide and aluminum oxide, to a high purity adsorbent powder in a wet mixture. The binder aids in forming or agglomerating the crystalline particles. The blended clay-adsorbent mixture may be extruded into cylindrical pellets or formed into beads which are subsequently calcined in order to convert the clay to an amorphous binder of considerable mechanical strength. The adsorbent may also be bound into irregular shaped particles formed by spray drying or crushing of larger masses followed by size screening. The adsorbent particles may thus be in the form of extrudates, tablets, spheres or granules having a desired particle range, preferably from about 16 to about 60 mesh (Standard U.S. Mesh) (1.9 mm to 250 microns). Clays of the kaolin type, water permeable organic polymers or silica are generally used as binders.

The active molecular sieve component of the adsorbent will preferably be in the form of small crystals present in the adsorbent particles in amounts ranging from about 75 to about 98-wt. % of the particle based on volatile-free composition. Volatile-free compositions are generally determined at 900° C., after the adsorbent has been calcined, in order to drive off all volatile matter. The remainder of the adsorbent will generally be the inorganic matrix of the binder present in intimate mixture with the small particles of the silicalite material. This matrix material may be an adjunct of the manufacturing process for the silicalite, for example, from the intentionally incomplete purification of the silicalite during its manufacture.

Those skilled in the art will appreciate that the performance of an adsorbent is often greatly influenced by a number of factors not related to its composition such as operating conditions, feed stream composition and the water content of the adsorbent. The optimum adsorbent composition and operating conditions for the process are therefore dependent upon a number of interrelated variables. One such variable is the water content of the adsorbent which is expressed herein in terms of the recognized Loss on Ignition (LOI) test. In the LOI test the volatile matter content of the zeolitic adsorbent is determined by the weight difference obtained before and after drying a sample of the adsorbent at 500° C. under an inert gas purge such as nitrogen for a period of time sufficient to achieve a constant weight. For the subject process it is preferred that the water content of the adsorbent results in an LOI at 900° C. of less than 7.0% and preferably within the range of from 0 to 4.0 wt. %.

An important characteristic of an adsorbent is the rate of exchange of the desorbent for the extract component of the feed mixture materials or, in other words, the relative rate of desorption of the extract component. This characteristic relates directly to the amount of desorbent material that must be employed in the process to recover the extract component from the adsorbent. Faster rates of exchange reduce the amount of desorbent material needed to remove the extract component, and therefore, permit a reduction in the operating cost of the process. With faster rates of exchange, less desorbent material has to be pumped through the process and separated from the extract stream for reuse in the process. Exchange rates are often temperature dependent. Ideally, desorbent materials should have a selectivity equal to about 1 or slightly less than 1 with respect to all extract components so that all of the extract components can be desorbed as a class with reasonable flow rates of desorbent material, and so that extract components can later displace desorbent material in a subsequent adsorption step.

U.S. Pat. No. 4,992,618 issued to S. Kulprathipanja, and which is incorporated by reference in its entirety, describes the use of a “prepulse” of a desorbent component in an SMB process for recovering normal paraffins. The prepulse is intended to improve the recovery of the extract normal paraffins across the carbon number range of the feed. The prepulse enters the adsorbent chamber at a point before (downstream) the feed injection point. A related SMB processing technique is the use of “zone flush.” The zone flush forms a buffer zone between the feed and extract bed lines to keep the desorbent e.g. normal pentane, from entering the adsorption zone. While the use of a zone flush requires a more complicated, and thus more costly rotary valve, the use of zone flush is preferred in the adsorption zones when high purity extract product are desired. In practice, a quantity of the mixed component desorbent recovered overhead from the extract and/or raffinate columns is passed into a separate splitter column. A high purity stream of the lower strength component of the mixed component desorbent is recovered and used as the zone flush stream. Further information on the use of dual component desorbents and on techniques to improve product purity such as the use of flush streams may be obtained from U.S. Pat. Nos. 3,201,491; 3,274,099; 3,715,409; 4,006,197 and 4,036,745 which are incorporated herein by reference in their entirety for their teaching on these aspects of SNB technology.

For purposes of this invention, various terms used herein are defined as follows. A “feed mixture” is a mixture containing one or more extract components and one or more raffinate components to be separated by the process. The term “feed stream” indicates a stream of a feed mixture which is passed into contact with the adsorbent used in the process. An “extract component” is a compound or class of compounds that is more selectively adsorbed by the adsorbent while a “raffinate component” is a compound or type of compound that is less selectively adsorbed. The term “desorbent material” shall mean generally a material capable of desorbing an extract component from the adsorbent. The term “raffinate stream” or “raffinate output stream” means a stream in which a raffinate component is removed from the adsorbent bed after the adsorption of extract compounds. The composition of the raffinate stream can vary from essentially 100% desorbent material to essentially 100% raffinate components. The term “extract stream” or “extract output stream” means a stream in which an extract material, which has been desorbed by a desorbent material, is removed from the adsorbent bed. The composition of the extract stream can vary from essentially 100% desorbent material to essentially 100% extract components.

At least portions of the extract stream and the raffinate stream are passed to separation means, typically fractional distillation columns, where at least a portion of desorbent material is recovered and an extract product and a raffinate product are produced. The terms “extract product” and “raffinate product” mean streams produced by the process containing, respectively, an extract component and a raffinate component in higher concentrations than those found in the extract stream and the raffinate stream withdrawn from adsorbent chamber. The extract stream may be rich in the desired compound or may only contain an increased concentration. The term “rich” is intended to indicate a concentration of the indicated compound or class of compounds greater than 50 mole percent.

It has become customary in the art to group the numerous beds in the SMB adsorption chamber(s) into a number of zones. Usually the process is described in terms of 4 or 5 zones. First contact between the feed stream and the adsorbent is made in Zone I, the adsorption zone. The adsorbent or stationary phase in Zone I becomes surrounded by liquid which contains the undesired isomer(s), that is, the raffinate. This liquid is removed from the adsorbent in Zone II, referred to as a purification zone. In the purification zone the undesired raffinate components are flushed from the void volume of the adsorbent bed by a material which is easily separated from the desired component by fractional distillation. In Zone III of the adsorbent chamber(s) the desired isomer is released from the adsorbent by exposing and flushing the adsorbent with the desorbent (mobile phase). The released desired isomer and accompanying desorbent are removed from the adsorbent in the form of the extract stream. Zone IV is a portion of the adsorbent located between Zones I and III which is used to segregate Zones I and III. In Zone IV desorbent is partially removed from the adsorbent by a flowing mixture of desorbent and undesired components of the feed stream. The liquid flow through Zone IV prevents contamination of Zone III by Zone I liquid by flow cocurrent to the simulated motion of the adsorbent from Zone III toward Zone I. A more thorough explanation of simulated moving bed processes is given in the Adsorptive Separation section of the Kirk-Othmer Encyclopedia of Chemical Technology at page 563. The terms “upstream” and “downstream” are used herein in their normal sense and are interpreted based upon the overall direction in which liquid is flowing in the adsorbent chamber. That is, if liquid is generally flowing downward through a vertical adsorbent chamber, then upstream is equivalent to an upward or higher location in the chamber.

In an SMB process the several steps e.g. adsorption and desorption, are being performed simultaneously in different parts of the mass of adsorbent retained in the adsorbent chamber(s) of the process. If the process was being performed with two or more adsorbent beds in a swing bed system then the steps may be performed in a somewhat interrupted basis, but adsorption and desorption will most likely occur at the same time.

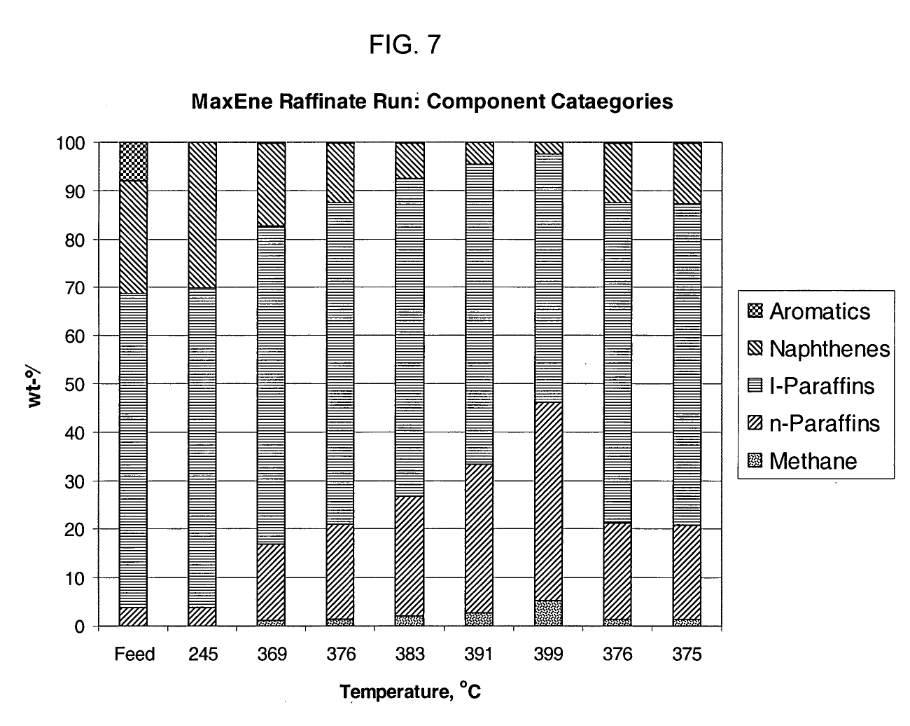

The aromatics contained in the naphtha feedstock, although generally amounting to less than the alkanes and cycloalkanes, may comprise from 2 to 20 mass % and more usually 5 to 10 mass % of the total. However, with the removal of the normal paraffins from the feedstream, the aromatic content is increased appreciably, and the efficiency of the process of converting aromatics to paraffins is increased. Benzene usually comprises the principal aromatics constituent of the preferred feedstock, optionally along with smaller amounts of toluene and higher-boiling aromatics within the boiling ranges described above. The adsorption unit separates the normal paraffins from the naphtha feedstream, and produces a raffinate stream rich in iso-paraffins, naphthenes, and aromatics. FIG. 7 shows the results of a ring opening reactor operated at different temperatures with a raffinate feedstream from an adsorption unit. As can be seen the aromatics are almost completely converted, there is a high conversion of naphthene and a substantial production of normal paraffins.

Naphtha feedstock and hydrogen comprise combined feed to the ring-opening unit, also known as the ring-cleavage zone, which contains a weakly acidic ring-cleavage catalyst and operates at suitable conditions to open naphthenic rings to form paraffins without a high degree of conversion to lighter products. The ring-cleavage catalyst comprises one or more platinum-group metals, selected from the group consisting of platinum, palladium, ruthenium, rhodium, osmium, and iridium, on a weakly acidic support comprising one or more of a refractory inorganic-oxide and a large-pore molecular sieve. The “weakly acidic support” has a substantial absence of acid sites, for example as an inherent property or through ion exchange with one or more basic cations.

The weak acidity of the ring-cleavage support may be determined using a variety of methods known in the art. A preferred method of determining acidity is the heptene cracking test as described below. Conversion of heptene, principally by cracking, isomerization and ring formation, is measured at specified conditions. Cracking is particularly indicative of the presence of strong acid sites. A weakly acidic catalyst suitable for ring cleavage demonstrates low conversion and particularly low cracking in the heptene test: conversion generally is less than 30% and cracking less than about 5%. The best supports demonstrate no more than about 5% conversion and negligible cracking.

The heptene cracking test also is effected in an atmospheric microreactor. In this test procedure an electrically heated reactor is loaded with 250 mg of 40-60 mesh particles made by crushing the sample particles. Each catalyst is dried in situ for 30 minutes at 200° C. using flowing hydrogen. The catalyst is then subjected to a reduction treatment for one hour at 550° C. in flowing hydrogen.

The reactor is then brought to the desired operational temperature of 425° C. (inlet). The feed stream to the reactor comprises hydrogen gas saturated with 1-heptene at 0° C. and ambient atmospheric pressure. The inlet temperature is held constant while the flow rate of the 1-heptene saturated hydrogen is varied in a predetermined pattern. Analysis is performed by analyzing the effluent using a gas chromatograph. Samples for analysis are automatically taken after 15 minutes of onstream operation at 250 cc/min. feed gas flow, at 45 minutes with the feed flowrate at 500 cc/min., at 75 minutes with the feed gas flowrate at 1000 cc/min., at 105 minutes with the feed gas flowrate at 125 cc/min. and after 135 minutes with the feed gas flowrate at the initial 250 cc/min. In each instance the feed gas flowrate is adjusted after the previous sample is taken. The analytical results are reported at each elapsed time during the test in weight percent indicating the composition of the effluent stream.

Alternatively, weak acidity may be characterized by the ACAC (acetonylacetone) test. ACAC is converted over the support to be tested at specified conditions: dimethylfuran in the product is an indicator of acidity, while methylcyclopentenone indicates basicity. Conversion over the support of the invention during a 5-minute period at 150° C. at a rate of 100 cc/min should yield less than 5 mass %, and preferably less than 1%, acid products. Conversion to basic products can usefully be in the range of 0-70 mass %.

Another useful method of measuring acidity is NH₃-TPD (temperature-programmed desorption) as disclosed in U.S. Pat. No. 4,894,142, incorporated herein by reference; the NH₃-TPD acidity strength should be less than about 1.0. Other methods such as ³¹P solids NMR of adsorbed TMP (trimethylphosphine) also may be used to measure acidity.

The preferred weakly acidic support optimally comprises a porous, adsorptive, high-surface-area inorganic oxide having a surface area of about 25 to about 500 m²/g. The porous support should also be uniform in composition and relatively refractory to the conditions utilized in the process. By the term “uniform in composition,” it is meant that the support be unlayered and relatively homogeneous in composition. Thus, if the support is a mixture of two or more refractory materials, the relative amounts of these materials will be constant and uniform throughout the entire support. It is intended to include within the scope of the present invention refractory inorganic oxides such as alumina, titania, zirconia, chromia, zinc oxide, magnesia, thoria, boria, silica-alumina, silica-magnesia, chromia-alumina, alumina-boria, silica-zirconia and other mixtures thereof.

The preferred refractory inorganic oxide for use in the present invention comprises alumina. Suitable alumina materials are the crystalline aluminas known as the theta-, alpha-, gamma-, and eta-alumina, with theta-, alpha-, and gamma-alumina giving best results. Magnesia, alone or in combination with alumina, comprises an alternative inorganic-oxide component of the catalyst and provides the required nonacidity. The preferred refractory inorganic oxide will have an apparent bulk density of about 0.3 to about 1.1 g/cc and surface area characteristics such that the average pore diameter is about 20 to 1000 angstroms, the pore volume is about 0.05 to about 1 cc/g, and the surface area is about 50 to about 500 m²/g.

The inorganic-oxide powder may be formed into a suitable catalyst material according to any of the techniques known to those skilled in the catalyst-carrier-forming art. Spherical carrier particles may be formed, for example, from the preferred alumina by: (1) converting the alumina powder into an alumina sol by reaction with a suitable peptizing acid and water and thereafter dropping a mixture of the resulting sol and a gelling agent into an oil bath to form spherical particles of an alumina gel which are easily converted to a gamma-alumina support by known methods; (2) forming an extrudate from the powder by established methods and thereafter rolling the extrudate particles on a spinning disk until spherical particles are formed which can then be dried and calcined to form the desired particles of spherical support; and (3) wetting the powder with a suitable peptizing agent and thereafter rolling the particles of the powder into spherical masses of the desired size. The powder can also be formed in any other desired shape or type of support known to those skilled in the art such as rods, pills, pellets, tablets, granules, extrudates, and like forms by methods well known to the practitioners of the catalyst material forming art.

The preferred form of carrier material for the ring-cleavage catalyst is a cylindrical extrudate. The extrudate particle is optimally prepared by mixing the preferred alumina powder with water and suitable peptizing agents such as nitric acid, acetic acid, aluminum nitrate, and the like material until an extrudable dough is formed. The amount of water added to form the dough is typically sufficient to give a Loss on Ignition (LOI) at 500° C. of about 45 to 65 mass %, with a value of 55 mass % being especially preferred. The resulting dough is then extruded through a suitably sized die to form extrudate particles.

The extrudate particles are dried at a temperature of about 150 to 200° C., and then calcined at a temperature of about 450 to 800° C. for a period of 0.5 to 10 hours to effect the preferred form of the refractory inorganic oxide.

It is essential that the catalyst be weakly acidic, as acidity in the zeolite lowers the selectivity to paraffins of the finished catalyst. The required weak acidity may be effected by any suitable method, including impregnation or ion exchange. Impregnation of one or more of the alkali and alkaline earth metals, especially potassium, in a salt solution is favored as being an economically attractive method. The metal effectively is associated with an anion such as hydroxide, nitrate or a halide such as chloride or bromide consistent with weak acidity of the finished catalyst, with a nitrate being favored. Optimally, the support is cold-rolled with an excess of solution in a rotary evaporator in an amount sufficient to provide a weakly acidic catalyst. The alkali or alkaline earth metal may be coimpregnated along with a platinum-group metal component, as long as the platinum-group metal does not precipitate in the presence of the salt of the alkali or alkaline earth metal.

Ion exchange is an alternative method of incorporating weak acidity into the catalyst. The inorganic-oxide support is contacted with a solution containing an excess of metal ions over the amount needed to effect weak acidity. Although any suitable method of contacting may be used, an effective method is to circulate a salt solution over the support in a fixed-bed loading tank. A water-soluble metal salt of an alkali or alkaline earth metal is used to provide the required metal ions; a potassium salt is particularly preferred. The support is contacted with the solution suitably at a temperature ranging from about 10 to about 100° C. Another suitable method comprises acid washing and steaming of the catalyst.

An alternative suitable support having inherent weak acidity may be termed a “synthetic hydrotalcite” characterized as a layered double hydroxide. Hydrotalcite is a clay with the ideal unit cell formula of Mg₆Al₂(OH)₁₆(CO₃)₄H₂O, and closely related analogs with variable magnesium/aluminum ratios may be readily prepared. W. T. Reichle has described in the Journal of Catalysis, 94, 547-557 (1985), the synthesis and catalytic use of such synthetic hydrotalcites, including materials having Mg and Al replaced by other metals. Calcination of such layered double hydroxides results in destruction of the layered structure and formation of materials which are effectively described as solid solutions of the resulting metal oxides.

These embodiments of the present support are disclosed in U.S. Pat. No. 5,254,743, incorporated by reference, and are solid solutions of a divalent metal oxide and a trivalent metal oxide having the general formula (M⁺²ₓO)(M⁺³_{y}O)OH_{y} derived by calcination of synthetic hydrotalcite-like materials whose general formula may be expressed as (M⁺²)ₓ(M⁺³)_{y}(OH)_{z}A_{q} rH₂O. M⁺² is a divalent metal or combination of divalent metals selected from the group consisting of magnesium, calcium, barium, nickel, cobalt, iron, copper and zinc. M⁺³ is a trivalent metal or combination of trivalent metals selected from the group consisting of aluminum, gallium, chromium, iron, and lanthanum. Both M⁺² and M⁺³ may be mixtures of metals belonging to the respective class: for example, M⁺² may be pure nickel or may be both nickel and magnesium, or even nickel-magnesium-cobalt; M⁺³ may be solely aluminum or a mixture of aluminum and chromium, or even a mixture of three trivalent metals such as aluminum, chromium, and gallium. A_{q} is an anion, most usually carbonate although other anions may be employed equivalently, especially anions such as nitrate, sulfate, chloride, bromide, hydroxide, and chromate. The case where M⁺² is magnesium, M⁺³ is aluminum, and A is carbonate corresponds to the hydrotalcite series.

It is preferable that the (M⁺²ₓO)(M⁺³_{y}O)OH_{y} solid solution has a surface area of at least about 150 m²/g, more preferably at least 200 m²/g and it is even more preferable that it be in the range from 300 to 350 m²/g. The ratio x/y of the divalent and trivalent metals can vary between about 2 and about 20, with the ratios of 2 to about 10 being preferred.

Methods of preparation are known in the art, and can be found in U.S. Pat. Nos. 5,811,624; 5,770,042; and 5,463,155 which are incorporated by reference in their entirety.

As with any of the catalysts used in the ring opening reactor, it is preferred that the catalyst also has an isomerization function for converting cyclohexane to methylcyclopentane (MCP). The isomerization of cyclohexane to MCP facilitates the ring opening function of the catalyst as it is easier to cleave a C₅ ring than a C₆ ring.

The ring-cleavage step of the present invention is observed to be particularly useful in combination with isomerization of light paraffins. By reducing the content of cyclics in the feed to the isomerization step, the proportion of catalyst available for isomerization of paraffins is increased.

Although hydrogen and light hydrocarbons may be removed by flash separation and/or fractionation from the paraffinic intermediate between the ring-cleavage zone and the isomerization zone, the intermediate preferably is transferred between zones without separation of hydrogen or light hydrocarbons. The exothermic saturation reaction provides a heated, paraffinic intermediate to the isomerization zone which generally requires no further heating to effect the required isomerization temperature. A cooler or other heat exchanger between the ring-cleavage zone and isomerization zone may be appropriate for temperature flexibility or for the startup of the process combination.

Contacting within the ring-cleavage and isomerization zones may be effected using the catalyst in a fixed-bed system, a moving-bed system, a fluidized-bed system, or in a batch-type operation. A fixed-bed system is preferred. The reactants may be contacted with the bed of catalyst particles in either upward, downward, or radial-flow fashion. The reactants may be in the liquid phase, a mixed liquid-vapor phase, or a vapor phase when contacted with the catalyst particles, with excellent results being obtained by application of the present invention to a primarily liquid-phase operation. The isomerization zone may be in a single reactor or in two or more separate reactors with suitable means therebetween to insure that the desired isomerization temperature is maintained at the entrance to each zone. Two or more reactors in sequence are preferred to enable improved isomerization through control of individual reactor temperatures and for partial catalyst replacement without a process shutdown.

Isomerization conditions in the isomerization zone include reactor temperatures usually ranging from about 40 to 250° C. Higher reaction temperatures are generally preferred in order to favor equilibrium mixtures having the highest concentration of normal alkanes. Temperatures in the range of about 150 to about 250° C. are preferred in the present invention. Reactor operating pressures generally range from about 100 kPa to 10 MPa absolute, preferably between about 0.5 and 4 MPa. Liquid hourly space velocities range from about 0.2 to about 15 volumes of isomerizable hydrocarbon feed per hour per volume of catalyst, with a range of about 0.5 to 5 hr⁻¹ being preferred.

Hydrogen is mixed with or remains with the paraffinic intermediate to the isomerization zone to provide a mole ratio of hydrogen to hydrocarbon feed of about 0.01 to 5. The hydrogen may be supplied totally from outside the process or supplemented by hydrogen recycled to the feed after separation from reactor effluent. Light hydrocarbons and small amounts of inserts such as nitrogen and argon may be present in the hydrogen. Water should be removed from hydrogen supplied from outside the process, preferably by an adsorption system as is known in the art. In a preferred embodiment the hydrogen to hydrocarbon mol ratio in the reactor effluent is equal to or less than 0.05, generally obviating the need to recycle hydrogen from the reactor effluent to the feed.

Water and sulfur are catalyst poisons especially for the chlorided platinum-alumina catalyst composition described hereinbelow. Water can act to permanently deactivate the catalyst by removing high-activity chloride from the catalyst, and sulfur temporarily deactivates the catalyst by platinum poisoning. Feedstock hydrotreating as described hereinabove usually reduces water-generating oxygenates to the required 0.1 ppm or less and sulfur to 0.5 ppm or less. Other means such as adsorption systems for the removal of sulfur and water from hydrocarbon streams are well known to those skilled in the art.

Any catalyst known in the art to be suitable for the isomerization of paraffin-rich hydrocarbon streams may be used as an isomerization catalyst in the isomerization zone. One suitable isomerization catalyst comprises a platinum-group metal, hydrogen-form crystalline aluminosilicate zeolite and a refractory inorganic oxide, and the composition preferably has a surface area of at least 580 m²/g. The preferred noble metal is platinum which is present in an amount of from about 0.01 to 5 mass % of the composition, and optimally from about 0.15 to 0.5 mass %. Catalytically effective amounts of one or more promoter metals preferably selected from Groups VIB(6), VIII(8-10), IB(11), IIB(12), IVA(14), rhenium, iron, cobalt, nickel, gallium and indium also may be present. The crystalline aluminosilicate zeolite may be synthetic or naturally occurring, and preferably is selected from the group consisting of FAU, LTL, MAZ and MOR with mordenite having a silica-to-alumina ratio of from 16:1 to 60:1 being especially preferred. The zeolite generally comprises from about 50 to 99.5 mass % of the composition, with the balance being the refractory inorganic oxide. Alumina, and preferably one or more of gamma-alumina and eta-alumina, is the preferred inorganic oxide. Further details of the composition are disclosed in U.S. Pat. No. 4,735,929, incorporated herein in its entirety by reference thereto.

A preferred isomerization catalyst composition comprises one or more platinum-group metals, a halogen, and an inorganic-oxide binder. Preferably the catalyst contains a Friedel-Crafts metal halide, with aluminum chloride being especially preferred. The optimal platinum-group metal is platinum which is present in an amount of from about 0.1 to 5 mass %. The inorganic oxide preferably comprises alumina, with one or more of gamma-alumina and eta-alumina providing best results. Optimally, the carrier material is in the form of a calcined cylindrical extrudate. The composition may also contain an organic polyhalo component, with carbon tetrachloride being preferred, and the total chloride content is from about 2 to 15 mass %. An organic-chloride promoter, preferably carbon tetrachloride, is added during operation to maintain a concentration of 30 to 300 mass ppm of promoter in the combined feed. Other details and alternatives of preparation steps and operation of the preferred isomerization catalyst are as disclosed in U.S. Pat. Nos. 2,999,074 and 3,031,419 which are incorporated herein by reference.

The isomerization zone generally comprises a separation section, optimally comprising one or more fractional distillation columns having associated appurtenances and separating lighter components from an isoparaffin-rich product. In addition, a fractionator may separate an isoparaffin concentrate from a cyclics concentrate with the latter being recycled to the ring-cleavage zone. Other techniques as taught in the art may be incorporated into the process combination to separate isoparaffin-rich product from recycle streams to ring cleavage and/or isomerization, including molecular-sieve adsorption or a combination of molecular-sieve adsorption and fractionation. One such embodiment comprises contacting the naphtha feedstock in the isomerization zone to obtain normal paraffin-rich product, separating the product by molecular-sieve adsorption at adsorption conditions to obtain normal paraffin concentrate and a cyclics concentrate containing isoparaffins, and converting the cyclics/isoparaffin concentrate in the ring-cleavage zone to produce paraffinic intermediate which is recycled to the isomerization zone. Alternatively the cyclics concentrate contains low-branched as well as normal paraffins, and optionally is fractionally distilled to separate a paraffinic recycle to isomerization and a cyclics stream to ring cleavage. Optional but non-limiting separation embodiments, including adsorption conditions and adsorbent characteristics, are disclosed in U.S. Pat. Nos. 4,585,826 and 5,043,525, incorporated by reference in their entirety.

Optionally, the isomerization process is incorporated within the ring opening reactor 50, and is amenable to the present invention. As shown in FIGS. 1, 4, and 5, the ring opening reactor 50 includes a catalyst for isomerization of the paraffins to increase the normal paraffin yield. The choice of catalysts may be affected as the operating conditions for the isomerization process will be in a temperature range from about 350 to about 425° C.

Further discussion and information concerning isomerization catalysts is available in U.S. Pat. Nos. 6,080,904; 5,382,731; 5,334,792; 4,834,866; and 4,783,575 which are incorporated by reference in their entirety.

### EXAMPLE

Simulations using a ring opening process on naphtha feedstreams show that the paraffin content will increase in the resulting process stream. Simulations using the ring opening process were performed to study the increase in light olefins from naphtha feed. Table 1 shows the results from simulations to compare yield estimates for light olefin production. The simulations were for a model naphtha feed, a commercial naphtha feed, a model cracker pyrolysis gas, and a model feed from a MaxEne process. MaxEne is a process for separating normal hydrocarbons from a hydrocarbon mixture and is licensed by UOP LLC.

TABLE 1
YIELD COMPARISONS

| | | Wt % | | | | Delta (to original Feed) | | | |
|---|---|---|---|---|---|---|---|---|---|
| | | Model | | Model | | Model | Commercial | Model | |
| | | Naphtha | Commercial | Cracker Py | Maxene | Naphtha | Naphtha | Cracker | Maxene |
| | | Feed | Naphtha Feed | gas | raffinate | Feed | Feed | Py gas | raffinate |
| Ethylene | Feed | 28.3 | 24.8 | 15.8 | 32.7 | 4.9 | 4.9 | 6.9 | 4.7 |
| | Post RO | 33.2 | 29.7 | 22.7 | 37.4 | | | | |
| Propylene | Feed | 12.7 | 9.2 | 7.2 | 10.6 | 2.2 | 4.3 | 5.4 | 1.8 |
| | Post RO | 14.9 | 13.5 | 12.6 | 12.4 | | | | |
| Methane | Feed | 15.3 | 14.2 | 15.8 | 16.6 | 3.9 | 6.4 | 6.9 | 4.1 |
| | Post RO | 19.2 | 20.6 | 22.7 | 20.7 | | | | |
| Butadiene | Feed | 5.3 | 4.6 | 4.8 | 4.6 | 0.1 | 0.1 | 0.0 | 0.2 |
| | Post RO | 5.4 | 4.7 | 4.8 | 4.8 | | | | |
| Heavies | Feed | 25.8 | 37.9 | 45.4 | 25.3 | −13.3 | −20.6 | −28.2 | −13.6 |
| | Post RO | 12.5 | 17.3 | 17.2 | 11.7 | | | | |

The calculations indicate that a 4-7% increase would be expected in ethylene production over the original feed and around a 2-6% increase would be expected in propylene production over the original feed. In addition, the simulations indicate the amount of heavies remaining would be expected to decrease from around 13-30% over the original feed. The tests indicate a substantial increase in the production of light olefins can be expected with the use of a ring opening reactor.

While the invention has been described with what are presently considered the preferred embodiments, it is to be understood that the invention is not limited to the disclosed embodiments, but is intended to cover various modifications and equivalent arrangements included within, the scope of the appended claims.

1. A process for improving the paraffin content of a feedstock to a steam cracking unit, comprising:
passing a feedstream comprising C₅ through C₉ hydrocarbons including C₅ through C₉ normal paraffins into a ring opening reactor, the ring opening reactor comprising a catalyst operated at conditions to convert aromatic hydrocarbons to naphthenes and a catalyst operated at conditions to convert naphthenes to paraffins, and producing a second feedstream; and
passing at least a portion of the second feedstream to a steam cracking unit.

2. The process of claim 1 further comprising:
passing the second feedstream to a first fractionation unit separating the second feedstream into a methane stream and a non-methane stream; and
passing the non-methane stream to the steam cracking unit.

3. The process of claim 1 further comprising passing a py-gas stream generated by the steam cracking unit to the ring opening reactor.

4. A process for improving the paraffin content of a feedstock to a steam cracking unit, comprising:
passing a feedstream comprising C₅ through C_{c} hydrocarbons including C₅ through C₉ normal paraffins into a first fractionation unit operated at conditions to separate the feedstream into a first process stream rich in C₅ paraffins and a second process stream comprising C₆ through C₉ hydrocarbons;
passing the second process stream into an adsorption unit, the adsorption unit comprising an adsorbent and operated at conditions to selectively adsorb normal paraffins, and produce a raffinate stream comprising non-normal C₆ through C₉ hydrocarbons;
passing at least a portion of the first process stream into the adsorption unit operated at desorption conditions to desorb the normal paraffins from the adsorbent, and produce an extract stream comprising normal C₆ through C₉ paraffins and C₅ paraffins;
passing the raffinate stream to a ring opening reactor where the raffinate stream is contacted with a catalyst for converting aromatic-hydrocarbons to naphthenes and a catalyst for converting naphthenes to paraffins at ring opening conditions to produce a ring opening process stream comprising n-paraffins and isoparaffins; and
passing the extract stream to a steam cracking unit, or passing the extract stream to a second fractionation unit operated at conditions to separate the extract stream into a third process stream comprising C₅ paraffins and a fourth process stream comprising C₆ through C₉ normal paraffins and passing the fourth process stream to the steam cracking unit and
passing at least a portion of the ring opening process stream to the steam cracking unit.

5. The process of claim 4 further comprising:
passing at least a portion of the ring opening process stream to a third fractionation unit operated at conditions to produce a methane stream and a non-methane stream; and
passing at least a portion of the non-methane stream to the steam cracking unit.

6. The process of claim 5 further comprising:
passing at least a portion of the non-methane stream to an isomerization unit operated at conditions to produce an effluent mixture comprising normal and iso-paraffins; and
passing the effluent mixture to the adsorption unit.

7. The process of claim 4 wherein the ring opening reactor comprises a catalyst having an acid function to convert cyclohexanes to cyclopentanes.

8. The process of claim 4 further comprising passing the feedstream through a hydrodesulfurization unit prior to passing the feedstream to the first fractionator.

9. The process of claim 4 further comprising passing a portion of the ring opening process stream into the first fractionation unit.

10. The process of claim 9 further comprising purging a portion of the ring opening process stream.

11. The process of claim 4 further comprising:
passing at least a portion of the ring opening process stream to a third fractionation unit, producing a methane stream and a non-methane stream; and
passing a portion of the non-methane stream to the first fractionation unit.

12. The process of claim 4 further comprising passing a py-gas stream generated by the steam cracking unit to the ring opening reactor.

13. A process for improving the paraffin content of a feedstock to a steam cracking unit, comprising:
passing a feedstream comprising C₅ through C₉ hydrocarbons including C₅ through C₉ normal paraffins into a ring opening reactor, the ring opening reactor comprising a catalyst for converting naphthenes to paraffins, and operated at ring opening conditions to produce a second feedstream;
passing the second feedstream into a first fractionation unit, operated at conditions to separate the feedstream into a first process stream rich in C₅ paraffins and a second process stream comprising C₆ through C₉ hydrocarbons;
passing the second process stream into an adsorption unit, the adsorption unit comprising an adsorbent and operated at conditions to selectively adsorb normal paraffins, and produce a raffinate stream comprising non-normal C₆ through C₉ hydrocarbons;
passing at least a portion of the first process stream into the adsorption unit operated at conditions to desorb the normal paraffins from the adsorbent, and produce an extract stream comprising normal C₆ through C₉ paraffins and C₅ paraffins; and
passing the extract stream to a steam cracking unit.

14. The process of claim 13 further comprising:
passing the raffinate stream to a second fractionation unit operated at conditions to separate the raffinate stream into a third process stream comprising non-aromatic hydrocarbons, and a fourth process stream comprising aromatic hydrocarbons; and
passing at least a portion of the third process stream to the steam cracking unit.

15. The process of claim 14 further comprising passing a portion of the fourth process stream to a reformer.

16. The process of claim 13 further comprising:
passing the raffinate stream to an isomerization unit operated at conditions to isomerize the non-normal paraffins to a mixture of n-paraffins and iso-paraffins, and produce a mixed paraffin stream;
passing the mixed paraffin stream to a third fractionation unit operated at conditions to separate the mixed paraffin stream into a fifth process stream comprising n-paraffins and a sixth process stream comprising iso-paraffins; and
passing the fifth process stream to the steam cracking unit.

17. The process of claim 16 further comprising passing a portion of the sixth process stream into the absorption unit.

18. The process of claim 13 further comprising passing a py-gas stream generated by the steam cracking unit to the ring opening reactor.

19. A process for improving the paraffin content of a feedstock to a steam cracking unit, comprising;
passing a feedstream comprising normal paraffins and non-normal hydrocarbons into an adsorption unit, the adsorption unit comprising an adsorbent and operated at conditions to selectively adsorb normal paraffins, and produce a raffinate stream comprising non-normal hydrocarbons;
passing a desorbent stream comprising a heavy normal hydrocarbon into the adsorption unit operated at desorption conditions to desorb the normal paraffins from the adsorbent, and produce an extract stream comprising the normal paraffins and the heavy normal hydrocarbon;
passing the extract stream to a fractionation unit operated at conditions to separate the extract stream into a steam cracker feedstream comprising the normal paraffins, and a desorbent recycle stream comprising the heavy normal hydrocarbon;
passing the steam cracker feedstream to a steam cracking unit;
passing the raffinate stream to a ring opening reactor where the raffinate stream is contacted with a catalyst for converting aromatic hydrocarbons to naphthenes and naphthenes to paraffins at ring opening conditions to produce a ring opening process stream comprising n-paraffins and iso-paraffins; and
passing a portion of the ring opening process stream to the steam cracking unit.

20. The process of claim 19 wherein the heavy normal paraffin is a normal paraffin having at least 12 carbons.

21. The process of claim 19 further comprising passing a py-gas stream generated by the steam cracking unit to the ring opening unit.

22. The process of claim 19 further comprising passing a portion of the ring opening process stream to an isomerization unit operated at conditions to produce an effluent mixture comprising normal and iso-paraffins.
